(19) United States
(12) Patent Application Publication
(10) Pub. No.: US 2022/0033225 A1
(43) Pub. Date: Feb. 3, 2022

(54) AIR AND SURFACE CLEANING SYSTEM FOR ENCLOSED SPACES, METHODS OF USE, AND METHODS OF MANUFACTURE

(71) Applicant: Henderson Building Solutions, LLC, Lenexa, KS (US)

(72) Inventors: Tracy E. Wagoner, Harrisonville, MO (US); David A. Goerke, Smithville, MO (US)

(21) Appl. No.: 17/391,558

(22) Filed: Aug. 2, 2021

Related U.S. Application Data
(60) Provisional application No. 63/059,358, filed on Jul. 31, 2020.

(51) Int. Cl. B66B 11/02; A61L 9/22; A61L 2/10

(52) U.S. Cl. CPC B66B 11/024; A61L 9/22; A61L 2202/25; A61L 2209/14; A61L 2202/11; A61L 2/10

(57) ABSTRACT

A system for active and automated cleaning of interior surfaces and air within an enclosed space. The system is generally composed of an air cleaning sub-system, a surface cleaning sub-system, and an interface sub-system that is hands-free to prevent contacting fomites and spreading disease. The air cleaning sub-system employs a fan mounted above the enclosed space. In an elevator environment, this can easily be installed to avoid structural crosshead beams. Air is then pushed down through a flexible boot which includes a HEPA filter, pushing filtered and ionized air down past individual's heads and exhausted through the bottom of the enclosed space, such as the toe kick vents in an elevator environment. In this way, the air reaching the individuals within the space is clean, fresh, and has not made contact with other individuals within the space prior to being inhaled.

### CROSS-REFERENCE TO RELATED APPLICATION

This application claims priority in U.S. Provisional Patent Application No. 63/059,358 Filed Jul. 31, 2020, which is incorporated herein by reference.

### BACKGROUND OF THE INVENTION

### 1. Field of the Invention

The present invention relates generally to an air and surface cleaning system and method for use thereof, and more specifically to automated systems and methods for providing clean air and cleaning surface areas of an enclosed space.

### 2. Description of the Related Art

Enclosed spaces, such as elevators, public transportation vehicles, and small rooms, require individuals to come into close proximity with others. This can lead to the spreading of diseases due to contaminated air or through contact with fomites such as door handles, elevator buttons, and other common surfaces. Existing systems, such as those common in elevators, pull air up through the bottom edge of the space and out through the top of the space. This can cause dust, smells, contaminants, viruses, and bacteria to be drawn up to individual's faces. What is needed is an improved way to clean the air and surfaces of such enclosed spaces actively and constantly so that such contaminants and disease-carrying elements can be prevented.

Heretofore there has not been available a system or method for cleaning systems for enclosed spaces with the advantages and features of the present invention.

### BRIEF SUMMARY OF THE INVENTION

The present invention generally provides multiple features for active and automated cleaning of interior surfaces and air within an enclosed space. The system is generally composed of an air cleaning sub-system, a surface cleaning sub-system, and an interface sub-system that is hands-free to prevent contacting fomites and spreading disease.

The air cleaning sub-system employs a fan, such as an adjustable 600 CFM fan, mounted above the enclosed space. In an elevator environment, this can easily be installed to avoid structural crosshead beams. Air is then pushed down through a flexible boot which includes a HEPA filter, pushing filtered and ionized air down past individual's heads and exhausted through the bottom of the enclosed space, such as the toe kick vents in an elevator environment. In this way, the air reaching the individuals within the space is clean, fresh, and has not made contact with other individuals within the space prior to being inhaled. In an elevator environment, the fan can be installed where the previous exhaust fan would have been, essentially reversing the way in which a typical elevator air handling system functions. An indicator light can be included to let individuals within the space know that the air cleaning system is active. Indicators can also be included to advise when to replace filters.

The surface cleaning sub-system employs 405 nm germicidal lighting within the enclosed space to provide continuous cleaning of the walls and other surfaces in the space. Lights such as the Indigo-Clean 405 nm Indigo Lights manufactured by Kenall Manufacturing Co. of Kenosha, Wis., can be used to replace existing lights within an enclosed space. The indigo blue color emitted from the system iRFndicates that the system is operational within the space, and a placard could be placed within the space to inform individuals that the system is actively cleaning surfaces. 405 nm light is needed to avoid the harmful effects of ultraviolet light, including UVC light, while still providing germicidal cleaning functionality.

The interface sub-system could be employed to prevent users from being required to touch surfaces to interact with the space. This interface sub-system would replace typical elements such as door handles, elevator buttons, or other interactive interfaces within an enclosed space. One example is the Toe-To-Go foot-activated elevator call button system created by Mad Elevator Inc. of Toronto, Canada. Similar foot-controlled or even voice-controlled interfaces can be used to replace typical touch-controlled interfaces to further prevent the transmission of diseases from surfaces in other enclosed spaces.

### BRIEF DESCRIPTION OF THE DRAWINGS

The drawings constitute a part of this specification and include exemplary embodiments of the present invention illustrating various objects and features thereof.

FIG. 1 is a three-dimensional isometric view of a prior art example of an enclosed space solution.

FIG. 2 is a three-dimensional isometric view of the present invention enclosed space solution.

FIG. 3 is a front sectional view thereof taken about the line of FIG. 2.

FIG. 4 is a flowchart diagramming the steps taken in practicing an embodiment of the present invention.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

### II. Prior Art Embodiment Enclosed Space System 52

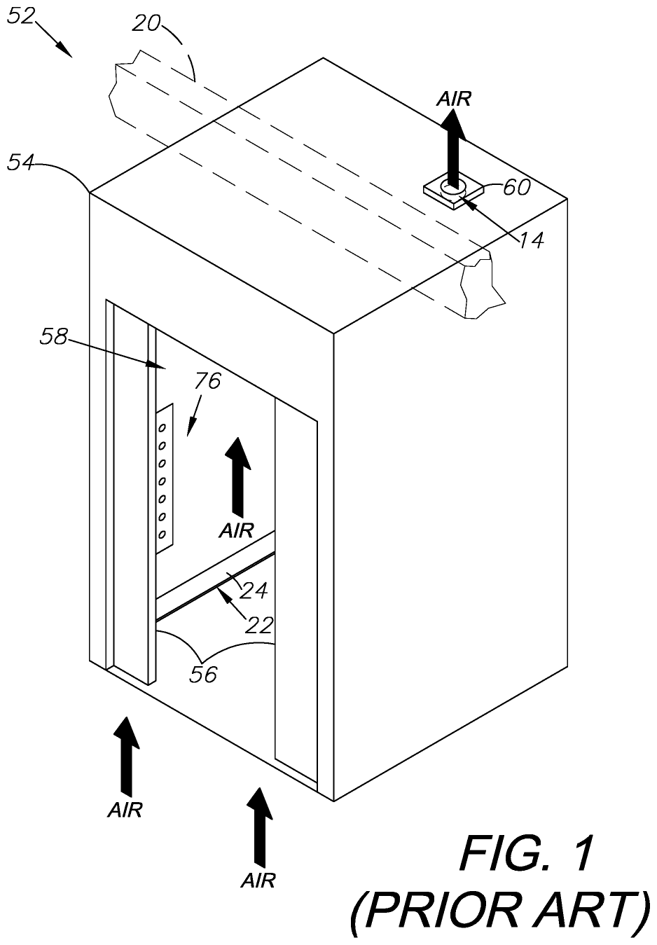

FIG. 1 shows the roof of a typical elevator car 54, functioning as an example enclosed space, with an exhaust fan 60 for drawing air up and out of the enclosed space within the elevator car 54. As is typical, the shaft and space around the elevator car where the air is being drawn into the car is dirty. The exhaust fan 60 pulls air up through vents 22 along the kick plate 24 within the elevator car, which can cause dust, smells, contaminants, viruses, and bacteria to be drawn up to individual's faces. Such systems are also commonly employed in other enclosed spaces, such as train cars, utility rooms, and other rooms both stationary and mobile. The exhaust fan often ceases to function due to structural elements 20 and unclean conditions.

Also shown in FIG. 1 is a prior art interface 76 within the entrance 58 of the elevator 54 through the doors 56 which require a user to physical contact with their hands. Existing light sources inside such enclosed spaces also fail to provide any additional functionality outside of providing illumination.

### III. Preferred Embodiment Enclosed Space System 2

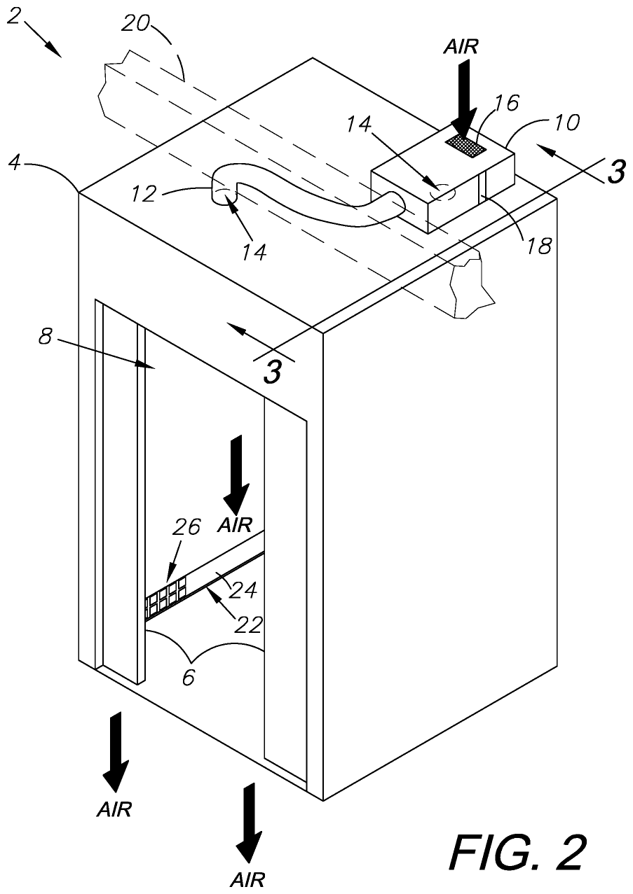

FIG. 2 shows an enclosed space of an elevator car 4 employing the enclosed space system 2 of the present invention. Unlike the prior art embodiment, air flow is instead directed from the top down through the vents 22 in the kick plate 24. Smells will not be drawn upwards toward the persons inside of the space, providing a cleaner and more present space.

The typical exhaust fan of the prior art embodiment is replaced with an air supply unit 10 with an air intake 16 and air filter 18 which pushes air down through the unit 10 through the hole 14 used previously for the exhaust fan, and therefore provides clean, filtered air from the head down in the enclosed space, with the air pushed out the bottom of the room at the end.

Unlike the exhaust fan, which might be blocked by structural elements 20, the air supply unit 10 can be used with a flexible hose duct 12 to connect to a remote hole 14 location in the room 4. Air is filtered through the air filter 18 and delivered through the hole 14 either directly or via such a flexible hose duct 12.

A 600 CFM fan, such as in a portable air purification system provided by SecureAire, LLC of Dunedin, Fla., can be installed directly into the already existing space where the exhaust fan exists after the exhaust fan is removed. In this way, air is filtered and ionized through the air purification system and is pushed down through the enclosed space where it would have previously been pulled up and out. The air is then pushed out through the same toe kick vents.

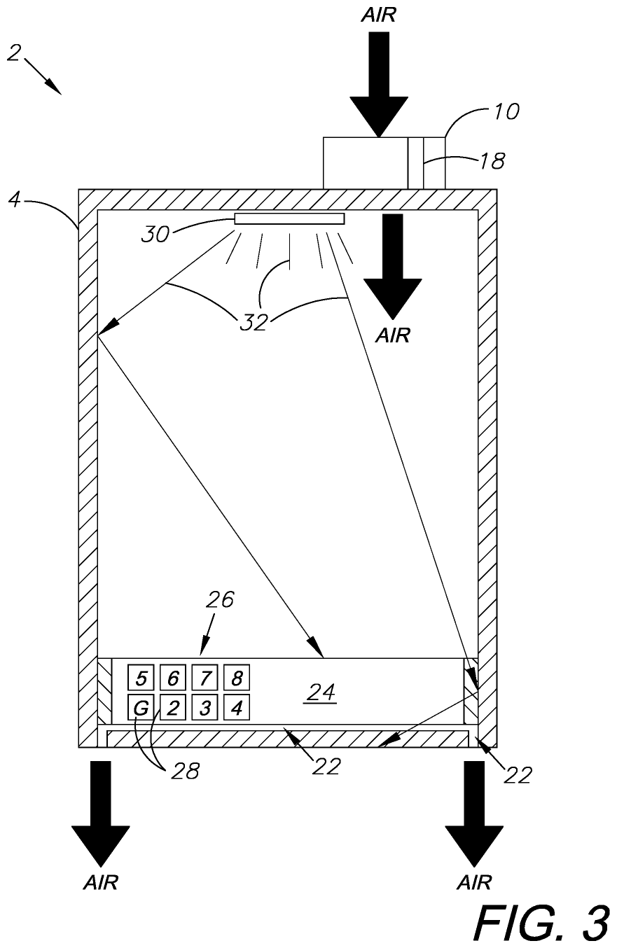

Additional clean-room elements can be included, such as germicidal light fixtures 30 shown in FIG. 3 which bounce light 32 off of surfaces, killing any harmful bacteria, viruses, or other elements living on the interior surfaces of the elevator car 4 interior including doors 6. Existing interior ceiling lights may use egg crate type ceilings with strip fluorescent fixtures which can easily be updated to use 405 nm light fixtures, such as the replacement the Indigo-Clean 405 nm Indigo LED Lights manufactured by Kenall Manufacturing Co. of Kenosha, Wis. Most, if not all, light fixtures similarly can be retrofit with such lights for constant active and automated cleaning of interior surfaces of the enclosed space.

In some situations and environments, it may be necessary to augment the lighting solutions within the enclosed space rather than retrofitting existing lights. Therefore, new light fixtures would need to be installed to properly provide the cleaning light functionality. This can be done using the same types of light fixtures and employing the same lighting elements to remove the harmful elements from the surfaces on the interior of the room.

FIG. 3 also shows how a hands-free interface system 26 can be employed to prevent individuals from skin-to-surface contacting surfaces within an enclosed space. The elevator buttons 76 are replaced with interactive elements 28 accessible with the user's feet, thereby preventing the spread of diseases caused by interacting with interfaces with a user's hands. Other non-contact interfaces could similarly be employed, including voice-controlled interfaces.

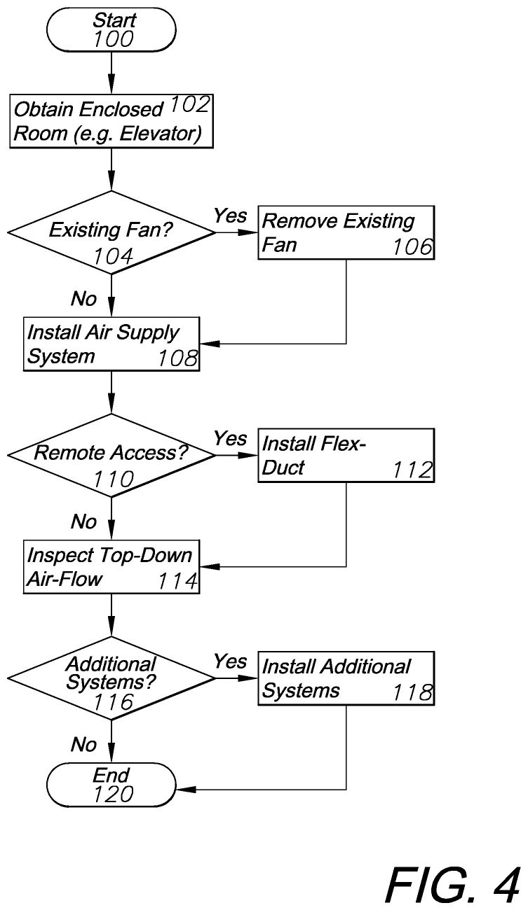

FIG. 4 shows the steps taken in practicing an embodiment of the present invention where the process begins at 100 and an enclosed room is obtained at 102. This room could be an existing enclosed space, such as a clean room, a laboratory, or an elevator car. These rooms could be constructed from scratch as well.

If there is an existing fan at 104, such as the exhaust fan shown in the prior art of FIG. 1, that fan is removed at 106. The air supply system is installed at 108 to provide forced air from the top of the room down. This unit can be installed on the existing holes for the exhaust fan, or new openings can be provided. If the hole requires remote access at 110, flexible boots can be used at 112 to extend the air supply system as needed.

The room is inspected for adequate air flow and air cleanliness at 114. If additional systems need to be installed at 116, those systems are installed at 118. This could include the light fixtures 30 or hands-free interface 26 disclosed above or other systems. The process ends at 120 with all components installed.

It is to be understood that while certain embodiments and/or aspects of the invention have been shown and described, the invention is not limited thereto and encompasses various other embodiments and aspects.

1. A system for providing cleaning to an enclosed space, the system comprising:
an air purification device comprising a filter, an inlet fan, and output fan, wherein the air purification device is installed externally to the enclosed space;
said air purification device configured to push air into said enclosed space from above, such that air passes from a top area within said enclosed space down through vents located near a bottom edge within said enclosed space;
said air purification device father configured to ionize said air; and
wherein said output fan is capable of providing at least 600 cubic feet of air per minute (CFM).

2. The system of claim 1, further comprising:
a light fixture configured to provide 405 nm wavelength light, said light fixture installed within an interior of said enclosed space such that said light fixture produces light which contacts interior walls of said enclosed space; and
wherein said light fixture further produces light which contacts an interface within said enclosed space.

3. The system of claim 1, further comprising:
a hands-free interface installed within said interior of said enclosed space; and
said hands-free interface comprising foot-activated elements configured to control at least one aspect of said enclosed space.

4. The system of claim 1, wherein said enclosed space comprises an elevator car, the system further comprising:
said vents located within a kick plate of the elevator car;
said air purification device configured to be installed onto an inlet passing through a roof of said elevator car; and
wherein said air purification device further configured to be installed to avoid structural elements above said elevator car.

5. The system of claim 4, further comprising:
a light fixture configured to provide 405 nm wavelength light, said light fixture installed within an interior of said enclosed space such that said light fixture produces light which contacts interior walls of said enclosed space;
wherein said light fixture further produces light which contacts interior surfaces within said enclosed space; and
wherein said air passes through said inlet and thorough said light fixture.

6. The system of claim 4, further comprising:
said inlet being located remotely from said air purification device; and
a flexible duct connecting said air purification device to said inlet.

7. A method of increasing cleanliness of an enclosed space, the method comprising the steps:
providing an enclosed space having a ceiling and a floor;
installing an air purification device above said ceiling of said enclosed space and connected to an inlet passing through said ceiling;
ionizing air with said air purification device; and
forcing said air down through said inlet from said air purification device into said enclosed space and out through vents located along said floor.

8. The method of claim 7, further comprising:
providing a light fixture installed onto said ceiling; and
providing 405 nm wavelength light with said light fixture, said light configured to contact interior surfaces within said enclosed space.

9. The method of claim 8, further comprising the step:
passing said air through said light fixture.

10. The method of claim 7, further comprising the step:
connecting said air purification device to said inlet via a flexible duct.

11. The method of claim 7, further comprising the steps:
providing a hands-free interface installed within said interior of said enclosed space; and
wherein said hands-free interface comprising foot-activated elements configured to control at least one aspect of said enclosed space.
